(19) United States
(12) Patent Application Publication
(10) Pub. No.: US 2007/0009431 A1
(43) Pub. Date: Jan. 11, 2007

(54) Solution and method for supporting imaging on a patient

(76) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/472,881

(22) Filed: Jun. 22, 2006

(30) Foreign Application Priority Data
Jun. 22, 2005 (DE) 10 2005 028 882.0

(51) Int. Cl. A61K 51/00; A61K 49/00

(52) U.S. Cl. 424/1.11; 424/9.1

(57) ABSTRACT

To support imaging on a patient, two different image recording methods are to be executed simultaneously. With each image recording method, a supporting fluid, for instance a contrast means or a tracer, can be introduced into the body of the patient. The invention proposes mixing the two supporting fluids with one another in a common solution, prior to releasing it into the patient, i.e. it provides a solution, which consists of a base solution without a medical agent, a first solution as a supporting fluid for a first image recording method and a second partial solution as a supporting fluid for a second image recording method. A plurality of combinations of the first and the second image recording methods are available. If necessary, a third partial solution can be present in the solution in order to simultaneously execute three image recording methods.

### CROSS REFERENCE TO RELATED APPLICATIONS

This application claims the benefits of German Patent application No. 10 2005 028 882.0 filed Jun. 22, 2005 and is incorporated by reference herein in its entirety.

### FIELD OF THE INVENTION

The invention relates to a solution (fluid) for supporting imaging on a patient with at least two image recording methods. It also relates to a method for supporting the imaging on a patient.

### BACKGROUND OF THE INVENTION

With medical examinations, different imaging methods are increasingly combined with one another. By way of example, a combination of an x-ray system and intravascular ultrasound (IVUS, described in detail for instance in EP 0 885 594 B1) is know for instance from U.S. Pat. No. 6,772,001 B2. The combination of MRI and an x-ray system is known from U.S. Pat. No. 6,263,043. A combination of computer tomography and PET is known for example from U.S. Pat. No. 5,391,877. First prototypes of a combination of computer tomography with an angiographic x-ray facility have been installed by Siemens.

A supporting fluid is used with numerous image recording methods of this type. In the x-ray field (with extracorporeal MRI and intravascular MRI, IVMRI) contrast means are administered to the patient in the blood. So-called tracers are used in nuclear medicine (with PET).

When an intended combination of image recording methods is required, such that two different supporting fluids are administered, these have hitherto been administered separately from one another. By way of example, infusions with infusions bottles are applied to the right and left arm of the patient.

### SUMMARY OF THE INVENTION

It is the object of the invention to facilitate the implementation of the image recording method.

For this purpose, the invention provides a solution and a method according to the claims.

The invention is based on the idea that the different partial solutions, which serve as supporting fluids for a respective image recording method, can be jointly administered to the patient in a mixture together with a base solution, because they already mix with one another in the blood of the patient.

The invention can be used across a wide range of applications. By way of example, the first image recording method can feature angiographic x-rays, extracorporeal radiation with ultrasound, computer tomography, MRI, PET or SPECT and the second image recording method can feature intravascular ultrasound, optical coherence tomography (OCT) or intravascular MRI. The first image recording method can also be computer tomography and the second image recording method can feature PET, SPECT or angiographic x-rays. Further, the first image recording method can be intravascular ultrasound and the second image recording method can be optical coherence tomography (OCT). The first image recording method can also be intravascular ultrasound and the second image recording method can be intravascular MRI. The first image recording method can also be MRI and the second image recording method can be angiographic x-rays, PET, SPECT or computer tomography.

These are all only examples. The invention is not restricted to the said methods, but can also be applied to potential combinations of image recording methods developing in the future.

A physiological saline solution is advantageously used as a base solution, the base solution can however also contain hydrochloric acid, trometamol, sodium calcium edetate and/or water.

The solution according to the invention can naturally also feature a third partial solution as a supporting fluid for a third image recording method, with the first image recording method then being angiographic x-rays for instance, the second image recording method being intravascular ultrasound and the third image recording method being optical coherence tomography (OCT).

The inclusion of a fourth and further partial solutions is also possible.

The solution according to the invention can also comprise a further partial solution containing a medicament for supporting body reactions arising from the first and/or second (and/or if necessary third) partial solution. These are medicaments which reduce the risk of potential negative body reactions, such as allergic reactions, shock and spasms, as are known in the prior art.

The solution generally also includes molecular or genetic markers and tracers (e.g. ED-B fibronectin to detect athersclerosis). It can also include nanoparticle contrast means or markers, ‘alphagammabeta3’ for instance. The fluorescent marker and/or contrast means are further included. ‘Target agents’ are also considered as partial solutions for the solution. These are diagnostic markers which accumulate in specific organs and tumors, for example Cancer Biomarker MUG-1.

It is also possible for instance for a mixture of different target agents to be produced for a cancer patient, with the one target agent accumulating in a primary tumor in the liver for instance and the second target agent accumulating in metastases in the spinal column.

The solution according to the invention is preferably bottled as a fluid in a storage container. Alternatively, it can also be provided in a syringe, where it can be injected intravenously or intra-arterially, or is also suited to oral or rectal ingestion or designed for inhalation. It can also be provided in an injector or an infusion device such as an infusion pump.

The solution can be supplied in accordance with the prior art, such that the packaging (the storage container or the syringe) is provided with a bar code label, and the container can comprise a radiation-impermeable protective shell such as a lead lining when radiopharmaceutics are used.

In accordance with the claimed method, at least two different imaging methods are determined to support the imaging on a patient, said imaging methods allowing the features of the patient to be mapped. A selection of the parameters for the first and the second imaging methods is carried out, whereupon quantities of supporting fluids can be defined for the first and the second imaging methods. The two quantities are mixed and the mixture is simultaneously introduced into the body of the patient. Imaging is subsequently carried out using the two imaging methods, with the two methods being carried out simultaneously or at least very soon after one another, in any case within the course of the same processes (diagnosis and/or treatment).

### BRIEF DESCRIPTION OF THE DRAWINGS

A preferred embodiment of the invention is now described with reference to the drawing, in which;

FIG. 1 shows a schematic representation of a situation, in which the solution according to the invention is administered to the patient.

### DETAILED DESCRIPTION OF THE INVENTION

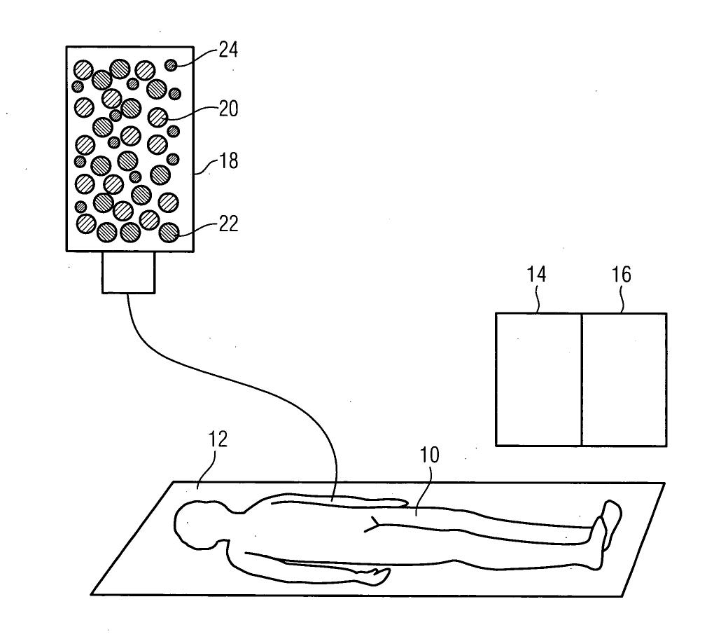

FIG. 1 shows a schematic representation of a patient 10. It has now emerged that an examination of the patient 10 is best carried out when two different imaging methods are implemented at the same time. The equipment 14 and 16 which is suited to implementing two imaging methods is located around the table 12 on which the patient lies, with boxes being schematically shown to symbolize corresponding apparatus. By way of example, the box 14 stands for an x-ray device for implementing digital subtraction angiography of the ventricle of the patient, and the box 16 stands for an apparatus for implementing double echocardiography on the heart beats or for detecting ventricle septal defects.

Both methods symbolized on the basis of apparatuses 14 and 16 require the administration of contrast means. Typical x-ray contrast means are Omnipaque®, Ultravist® und Biliscopin® (all by Schering AG). Typical ultrasound contrast means are Levovist®, Echovist®-200 and Echovist®-300 (all by Schering AG) as well as Optison, Echogen, Sonovue, Aerosomes, Imagent, NC 100100, Quantison and Sonovist.

By virtue of the settings of the apparatuses 14 and 16, the x-ray contrast means Omnipaque® in a quantity of 30 ml is suitable as x-ray contrast means, and 14 ml of Echovist® is suitable as ultrasound contrast means, with the milliliters referring to the medicinally effective solution. These two solution are mixed with one another in an infusion bottle 18 and are shown in FIG. 1 as differently shaded balls (molecules), see the 30 ml Omnipaque®, which are indicated with the reference character 20, the 14 ml Echovist®, which are indicated with the reference character 22. The infusion bottle 18 further comprises 6 ml of pharmaceutical exipient, physiological saline solution for instance, which is labelled 24.

This produces a 50 ml infusion bottle, in which the x-ray contrast means and the ultrasound contrast means are both located and which is suitable for direct administration to the patient 10. Before the beginning of one of the two image recording methods, the solution is administered to the patient 10 from the infusion bottle 18. Once sufficient solution is provided in the bloodstream of the patient, the actual examination can start. During the examination, images can be recorded simultaneously using the apparatuses 14 and 16, by means of which features of the patient can be mapped. The digital subtraction angiography of the ventricle can thus take place simultaneously with or very soon after the echocardiography of the heart beats. As both agents are present at the same time, a more rapid change between the two operators 14 or 16 is possible or the simultaneous use thereof.

The invention is, as previously mentioned, not restricted to the combination of x-ray imaging with ultrasound imaging. Further partial solutions can be found in the infusion bottle. By way of example, Isovist® 240/-300 by Schering AG or Gastrografin® can be used for computer tomographs, and both Resovist® and Gadovist® by Schering AG or also Magnevist® can be used with extracorporeal MRI as well as with intravascular MRI.

For nuclear medical examinations, for instance PET examinations, the following contrast means and/or tracers can be used: FDG(F18-desoxyglucose), 6-[18F]fluoro-L-dopa, 16alpha-[18F]fluoroestradiol, [15O]water, [13N]ammonia und [11C]acetate.

The invention is advantageous in that only one catheter and/or one access path must be placed in the body to be examined, in order to achieve an improved image quality, in particular of the contrast behavior, in the case of an examination using different imaging methods. The diagnosis and treatment for the patients can thus be carried out in a more reliable and rapid manner and with improved quality.

1-11. (canceled)

12. A medicinal solution that assists medical imaging of a patient undergoing a plurality of different image recording methods, comprising:
a base solution that does not contain a medical image improvement agent;
a first partial solution that improves the recorded image quality of a first image recording method; and
a second partial solution that improves the recorded image quality of a second image recording method.

13. The solution according to claim 12, wherein:
the first image recording method is selected from the group consisting of: angiographic x-rays, extracorporeal acoustic irradiation with ultrasound, computer tomography, MRI, PET and SPECT, and
the second image recording method is selected from the group consisting of: intravascular ultrasound, optical coherence tomography (OCT) and intravascular MRI.

14. The solution according to claim 12, wherein:
the first image recording method is computer tomography and the second image recording method is selected from the group consisting of: PET, SPECT and angiographic x-rays, or
the first image recording method is intravascular ultrasound and the second image recording method is optical coherence tomography (OCT), or
the first image recording method is intravascular ultrasound and the second image recording method is intravascular MRI, or
the first image recording method is MRI and the second image recording method is selected from the group consisting of: angiographic x-rays, PET, SPECT and computer tomography.

15. The solution according to claim 12, wherein the base solution is selected from the group consisting of: physiological saline solution, hydrochloric acid, trometamol, natrium calcium edentate, water and combinations thereof.

16. The solution according to claim 15 wherein the first and second supporting fluids are a contrast enhancing medium, a marker or a tracer.

17. The solution according to claim 12, further comprising a third partial solution that improves the quality of the recorded image for a third image recording method.

18. The solution according to claim 17, wherein the first image recording method is angiographic x-rays, the second image recording method is intravascular ultrasound, and the third image recording method is optical coherence tomography (OCT).

19. The solution according to claim 17, further comprising a fourth partial solution that improves the quality of the recorded image for a fourth image recording method.

20. The solution according to claim 19, further comprising a partial solution that contains a medicament that suppresses bodily reactions arising from the first, second, third or fourth partial solution.

21. The solution according to claim 20, wherein the solution is provided in a syringe, an injector or an infusion pump.

22. A method for improving the recording of medical images of a patient, comprising:
determining a plurality of different image recording methods that record images of internal features of the patient;
selecting a plurality of parameters for a first image recording method;
selecting a plurality of parameters for a second image recording method;
selecting a quantity of a first partial fluid that improves the imaging for the first image recording method;
selecting a quantity of a second partial fluid that improves the imaging for the second image recording method;
mixing the first and second supporting fluids;
introducing the first and second supporting fluid mixture into the body of the patient; and
recording images of the patient via the two image recording methods in close time proximity to each other.

23. The method as claimed in claim 22, wherein the first and second image recording methods are performed one after another.

24. The method as claimed in claim 22, wherein the first and second image recording methods are performed during the same medical procedure or examination.

25. An improved method for recording internal medical images of a patient, comprising:
determining a plurality of different image recording methods that record images of internal features of the patient, wherein the image recording methods are selected from the group consisting of: angiographic x-rays, extracorporeal acoustic irradiation with ultrasound, computer tomography, MRI, PET, SPECT, intravascular ultrasound, optical coherence tomography (OCT) and intravascular MRI;
selecting a plurality of parameters for a first image recording method and a second image recording method;
selecting a quantity of a first partial fluid that improves the imaging for the first image recording method and a quantity of a partial supporting fluid that improves the imaging for the second image recording method, wherein the first and second supporting fluids are selected from the group consisting of: contrast enhancing medium, a marker or a trace;
introducing the first and second supporting fluids into the body of the patient; and
recording images of the patient via the two image recording methods in close time proximity to one another.
